(19) United States
(12) Patent Application Publication
(10) Pub. No.: US 2010/0122698 A1
(43) Pub. Date: May 20, 2010

(54) NEONATAL AIRWAY STENT

(75) Inventors: Thomas H. SHAFFER, Chadds Ford, PA (US); Marla R. Wolfson, Wyndmoor, PA (US); Thomas L. Miller, Woodstown, NJ (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: The Nemours Foundation, Jacksonville, FL (US)

(21) Appl. No.: 12/273,871

(22) Filed: Nov. 19, 2008

(51) Int. Cl. A61F 2/04; A61M 16/00

(52) U.S. Cl. 128/204.18; 623/23.7; 606/151

(57) ABSTRACT

An adjustable neonatal airway stent for use in the airways of a neonate is provided. The stent may include a flexible mesh member having a generally cylindrical shape and size that is capable of being delivered to a neonatal airway. The mesh member may include overlapping inner and outer portions, where the inner portion is adjacent to an inner edge of the flexible mesh. Further, the stent may include a plurality of locking projections that extend outwardly from the inner portion and are capable of engaging the mesh of the overlapping outer portion to prevent contraction of the stent, while allowing expansion of the stent to at least one diameter when implanted in a neonatal airway. The projections may include angularly extending tips, and the stent may be expandable to a second diameter greater than the first diameter in situ.

### BACKGROUND OF THE INVENTION

1. Field of the Invention

The invention relates generally to devices and methods of employing the device for expanding and maintaining the patency of tubular body airways, and more particularly, to an expandable stent for supporting the large airways in neonates.

2. Related Art

The large conducting airways are complex organs within the pulmonary system. C-shape cartilage rings provide support and maintain the shape of the airway. Cellular and connective tissue layers within these rings perform various functions, including conditioning inspired air, movement of cilia, and production of mucus.

In premature infants, or infants subjected to mechanical respiratory support, the airways are often structurally compromised. Efforts to ventilate the lung, either spontaneously or mechanically, may result in airway structural collapse or deformity. In addition, mechanical ventilation may result in denudation of the airway mucosal layer. Early airway insult has been associated with chronic lung disease including the development of airway reactivity and asthmatic symptoms.

Stents are widely used in the medical field and have been used in the adult population to treat tracheal stenosis and vascular disease.

U.S. Pat. No. 5,007,926 (Derbyshire) discloses an expandable stent for large airways and other body ducts, comprising a mesh made from woven longitudinal and transverse wires, rolled into a cylindrical form such that the edge portions overlap each other. The stent is maintained in its expanded state by the interaction between the frayed ends of the transverse wires at one edge of the sheet with a portion of overlapping mesh. These wire ends engage the mesh sheet to prevent collapse of the stent but do not grasp or grab the wire mesh to hold the overlapping edge portions together. Further, because this limited mechanical connection between the inner and outer overlapping portions of the mesh is via a single row of protruding wire tips at one end of the mesh, this stent may lack the necessary intimate contact between inner overlapped portion and the opposing lumen wall to facilitate and enable full epithilialization of the stent in an airway. Thus, this stent may be more likely to migrate in the airway, blocking the cilia from performing their normal functions, and providing less protection of the mucosal layer of the airway from denudation.

Similarly, U.S. Pat. No. 5,443,500 (Sigwart) discloses an intravascular stent made from a perforated sheet forming an open reticulated lattice structure. The stent is held in its expanded state by a single row of integrated holding flaps at one edge of the rolled sheet which protrude into the lattice openings of the overlapping sheet but do not grasp or otherwise prevent separation between the overlapping portions of the sheet. In addition, because Sigwart is concerned with preventing damage to blood vessel walls, it specifically teaches away from a fiber mesh construction in favor of a smooth perforated sheet that will be more likely to migrate and less likely to encourage epithilialization.

U.S. Pat. No. 5,578,075 (Dayton) discloses an expandable stent for body conduits such as blood vessels, urethra, and bile ducts. The stent is formed from a perforated solid sheet of stainless steel or nitinol rolled into a cylindrical form. The stent is held in an expanded position by rounded or pointed tabs that protrude into the perforations of the overlapping sheet but do not grasp or latch onto the overlapping sheet. The course spacing of perforations in the stent enables only a discrete or quantized expansion of the stent from one diameter to the next. Additionally, the open space fraction of the perforated sheet structure is much less than that of a wire mesh, thus inhibiting epithilialization and cilial function.

U.S. Pat. No. 5,824,054 (Khosravi) discloses an expandable stent for body lumens made from a lattice sheet of nitinol having teeth at one end which fit into the lattice openings of the overlapping rolled sheet to hold the stent at a particular size. This stent suffers from the shortcomings of having teeth that do not grasp or grab the overlapping sheeting, as well has having only a single row of teeth securing the inner and outer overlapped portions. Additionally, this stent has the decreased epithilialization capabilities of a perforated sheet with a relatively small open space fraction.

U.S. Pat. No. 5,423,885 (Williams) discloses an expandable stent for body lumens such as blood vessels. The stent is formed from a rolled sheet of stainless steel, nitinol, platinum, tantalum, or gold. Angled, V-shaped teeth engage matching openings in the sheet to lock the stent at a particular diameter and to prevent its collapse to a smaller state. These teeth do not grasp or latch onto the overlapping sheet. While the pointed teeth of this stent may provide anchoring of the stent in the lumen, epithilialization and cilial function would be greatly inhibited by the very small open space fraction of the perforated sheet design.

It can be seen from the foregoing discussion that existing stents are not suited for use in the airways of neonates. There is a need for an adjustable, airway stent particularly adapted for use in the neonatal environment.

### SUMMARY OF THE INVENTION

The invention provides an adjustable neonatal airway stent having a large dilation range that can be tightly rolled for insertion into the small lumen and may be successively adjusted as the lumen grows. The stent may protect the mucosal lining from denudation during forced respiration, and encourage airway growth. Bioactive materials such as drugs may be incorporated into the stent to achieve therapeutic effects. A time release mechanism may be used for delivery of the therapeutic material, such as human growth factor, stem cells and other materials.

The neonatal airway stent may be expandable by any means known in the art including, e.g., self-expandable, balloon-expandable, thermal activation, etc. In any case, the neonatal airway stent may additionally be employed to encourage airway growth by being expanded via a tracheal balloon to exert distending pressuring outwardly against the airway.

The invention may be implemented in a variety of ways. According to one aspect of the invention, an adjustable neonatal airway stent is provided. The stent may include a flexible mesh member having a generally cylindrical shape and size capable of being delivered to a neonatal airway and may include overlapping inner and outer portions such that the inner portion is adjacent to an inner edge of the flexible mesh. Moreover, the stent may include a plurality of locking projections that extend outwardly from the inner portion and engage the overlapping outer portion to prevent contraction of the stent but allow expansion of the stent to at least one first diameter when implanted in a neonatal airway and permit the stent to expand in situ to a second diameter greater than the first diameter. The projections may include angularly extending tips. The locking projections may be disposed on the mesh inner portion at a distance from the mesh inner edge. The locking projections may be, for example, one-way locking projections, two-way locking projections, barbs, teeth, tabs, and fingers. The stent may have a diameter in the range of about 1.5 mm to about 6 mm. Specifically, the stent may have a diameter of about 1.5 mm for primary airways and a diameter of about 6 mm for secondary airways.

The flexible mesh member and the locking projections may be composed of biocompatible or hypoallergenic metal or plastic. The flexible mesh member and the locking projections may be composed of a biabsorbable material. The locking projections may be disposed in at least one row generally parallel to the mesh inner edge. The locking projections may be disposed in at least two rows generally parallel to the mesh inner edge. The locking projections may be disposed in an irregular array on the overlapping mesh inner portion. The locking projections may be disposed along the mesh inner edge.

The flexible mesh member and the locking projections may be coated with a bioactive agent to be delivered to the airway of a neonate. The bioactive agent may be one or more compounds selected from the group consisting of an adrenocortical steroid, an adrenocortical suppressant, an analgesic, an anti-asthmatic, an antifungal, an antihistaminic, an anti-infective, an anti-inflammatory, an antineoplastic, an antiviral, a bronchodilator, a glucocorticoid, a vasoconstrictor, a vasodilator, a growth factor, and stem cells.

According to another aspect of the invention, a method of protecting the mucosal layer of an airway in a neonate from denudation due to mechanical ventilation is provided. The method may include providing an adjustable stent including a flexible mesh member having a plurality of locking projections extending outwardly from the mesh portion, and disposed about a portion of one side of the mesh member adjacent to an edge thereof, rolling the mesh member into a collapsed position having a generally cylindrical shape and size having overlapping inner and outer portions, such that the mesh may be capable of fitting within a neonatal airway, retaining the mesh member in the collapsed position, delivering the stent into the airway of the neonate, positioning the stent in the desired region in the airway, and implanting the stent in an expanded position that is in contact with the airway so that the locking projections engage another section of the overlapping mesh outer portion to retain the stent in the expanded state. The method may further include inserting a balloon catheter within the cylindrically shaped mesh member, pressurizing the balloon catheter to expand the stent to a further expanded state so that the locking projections engage the mesh of the overlapping mesh outer portion to retain the stent in the further expanded state, and depressurizing and removing the balloon catheter from the airway.

The locking projections may be disposed on the mesh portion at a distance from the edge. The locking projections may be disposed in at least one row on the overlapping mesh inner portion generally parallel to the mesh member edge when the mesh member is rolled up. The locking projections may be disposed in an array on the overlapping mesh inner portion when the mesh member is rolled up. The locking projections may be disposed along the mesh member edge.

In a yet a further aspect of the invention, a method of enhancing growth of an underdeveloped airway in a neonate is provided. The method may include providing an adjustable diameter stent having a flexible mesh member including a plurality of locking projections extending outwardly from the mesh portion, and disposed about a portion of one side of the mesh member adjacent to an edge thereof. Rolling the mesh member into a collapsed position having a generally cylindrical shape and size with overlapping inner and outer portions such that the mesh is capable of fitting within a neonatal airway, retaining the mesh member in the collapsed position, delivering the stent into the airway of the neonate, positioning the stent where desired in the airway, implanting the stent in an expanded position in contact with the airway so that the locking projections engage the mesh of the overlapping mesh outer portion to retain the stent in the expanded state, inserting a balloon catheter within the cylindrically shaped mesh member, pressurizing the balloon catheter to expand the stent to a further expanded state so that the locking projections engage another section of the overlapping mesh outer portion to retain the stent in the further expanded state, depressurizing and removing the balloon catheter from the airway, and temporarily re-inserting and pressurizing the balloon catheter to exert sufficient outward pressure on the cylindrical stent to distend the airway of the neonate without injury, thereby encouraging the growth thereof. The projections may be disposed on the mesh portion at a distance from the edge.

Additional features, advantages, and embodiments of the invention may be set forth or apparent from consideration of the following detailed description, drawings, and claims. Moreover, it is to be understood that both the foregoing summary of the invention and the following detailed description are exemplary and intended to provide further explanation without limiting the scope of the invention claimed. The detailed description and the specific examples, however, indicate only preferred embodiments of the invention. Various changes and modifications within the spirit and scope of the invention will become apparent to those skilled in the art from this detailed description.

### BRIEF DESCRIPTION OF THE DRAWINGS

The accompanying drawings, which are included to provide a further understanding of the invention, are incorporated in and constitute a part of this specification, illustrate embodiments of the invention and together with the detailed description serve to explain the principles of the invention. No attempt is made to show structural details of the invention in more detail than may be necessary for a fundamental understanding of the invention and various ways in which it may be practiced. In the drawings:

FIG. 1 is a perspective, schematic view of one embodiment of a neonatal airway stent constructed according to the principles of the invention in a generally rolled up position.

FIG. 2 is a greatly enlarged, schematic top plan view of the neonatal airway stent of the invention schematically showing ratcheting barbs disposed in an overlapping portion of the stent.

FIG. 3 is a perspective, schematic view of the neonatal airway stent of the invention in a rolled up position.

FIG. 4 is a perspective, schematic view of another embodiment of the neonatal airway stent of the invention showing the barbs disposed in staggered rows at a distance from the inner axial edge of the stent.

FIG. 5 is a perspective, schematic view of another embodiment of the neonatal airway stent of the invention showing the barbs disposed along the inner axial edge of the stent.

FIG. 6 is a perspective, schematic view of another embodiment of the neonatal airway stent of the invention showing the barbs disposed in staggered rows disposed along the inner axial edge and disposed at a distance from the inner axial edge of the stent.

FIG. 7 is a perspective, schematic view of yet another embodiment of the neonatal airway stent of the invention showing the barbs disposed in a single row, but at various angles towards the inner axial edge of the stent.

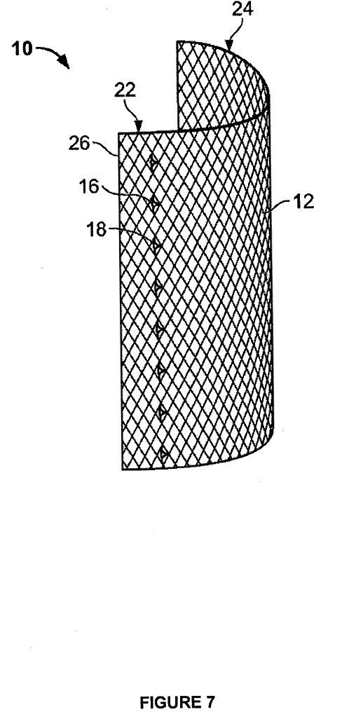

FIG. 8 is a perspective, schematic view of yet another embodiment of the neonatal airway stent of the invention showing the barbs disposed in an irregular array about the inner edge portion and along the inner axial edge of the stent.

FIG. 9 is a perspective, schematic view of an additional embodiment of the neonatal airways stent of the invention illustrating a generally unrolled position showing the barbs disposed in an irregular array about the inner edge portion.

FIG. 10 is a perspective, schematic cross-sectional view of a neonatal airway stent of the invention.

### DETAILED DESCRIPTION OF THE INVENTION

It is understood that the invention is not limited to the particular methodology, protocols, and reagents, etc., described herein, as these may vary as the skilled artisan will recognize. It is also to be understood that the terminology used herein is used for the purpose of describing particular embodiments only, and is not intended to limit the scope of the invention. It also is be noted that as used herein and in the appended claims, the singular forms “a,” “an,” and “the” include the plural reference unless the context clearly dictates otherwise. Thus, for example, a reference to “a lesion” is a reference to one or more lesions and equivalents thereof known to those skilled in the art.

Unless defined otherwise, all technical and scientific terms used herein have the same meanings as commonly understood by one of ordinary skill in the art to which the invention pertains. The embodiments of the invention and the various features and advantageous details thereof are explained more fully with reference to the non-limiting embodiments and examples that are described and/or illustrated in the accompanying drawings and detailed in the following description. It should be noted that the features illustrated in the drawings are not necessarily drawn to scale, and features of one embodiment may be employed with other embodiments as the skilled artisan would recognize, even if not explicitly stated herein. Descriptions of well-known components and processing techniques may be omitted so as to not unnecessarily obscure the embodiments of the invention. The examples used herein are intended merely to facilitate an understanding of ways in which the invention may be practiced and to further enable those of skill in the art to practice the embodiments of the invention. Accordingly, the examples and embodiments herein should not be construed as limiting the scope of the invention, which is defined solely by the appended claims and applicable law. Moreover, it is noted that like reference numerals reference similar parts throughout the several views of the drawings.

Moreover, provided immediately below is a “Definition” section, where certain terms related to the invention are defined specifically. Particular methods, devices, and materials are described, although any methods and materials similar or equivalent to those described herein can be used in the practice or testing of the invention. All references referred to herein are incorporated by reference herein in their entirety.

The terms “active agent,” “drug,” “therapeutic agent,” and “pharmacologically active agent” are used interchangeably herein to refer to a chemical material or compound which, when administered to an organism (human or animal) induces a desired pharmacologic effect. Included are derivatives and analogs of those compounds or classes of compounds specifically mentioned that also induce the desired pharmacologic effect. In particular, the therapeutic agent may encompass a single biological or abiological chemical compound, or a combination of biological and abiological compounds that may be required to cause a desirable therapeutic effect.

By the terms “effective amount” or “therapeutically effective amount” of an agent as provided herein are meant a nontoxic but sufficient amount of the agent to provide the desired therapeutic effect. The exact amount required will vary from subject to subject, depending on the age, weight, and general condition of the subject, the severity of the condition being treated, the judgment of the clinician, and the like. Thus, it is not possible to specify an exact “effective amount.” However, an appropriate “effective” amount in any individual case may be determined by one of ordinary skill in the art using only routine experimentation.

The term “bioabsorbable” as used herein generally may include a bioabsorbale material such as poly-D, L-lactic acid, polyethylene glycol, polydioxanone, polylactic acid, 70L/30DL polylactide, polyglycolide, poly(orthoester), calcium sodium metaphosphate, hydroxyapatite, calcium phosphate, polytetra fluoroethylene, collagen I, II, IX, X, and XI, durapatite, and hydrogel.

The terms “treating” and “treatment” as used herein refer to reduction in severity and/or frequency of symptoms, elimination of symptoms and/or underlying cause, prevention of the occurrence of symptoms and/or their underlying cause, and improvement or remediation of damage. Thus, for example, the present method of “treating” individuals afflicted with conditions that compromise airways, as the term “treating” is used herein, encompasses treatment of conditions that compromise airways in a clinically symptomatic individual.

The terms “condition,” “disease” and “disorder” are used interchangeably herein as referring to a physiological state that can be detected, prevented or treated by the surgical techniques, devices and/or therapeutic agent as described herein. Exemplary diseases and conditions in which the stent system, methods, and therapeutic agents of the invention may be used may include, but are not limited to, obstructed airways caused by tracheal stenosis, anastomotic stenosis after lung transplantation, and other benign and/or malignant conditions compromising the airways tracheal stenosis and stenosis, for example.

The term “patient” as in treatment of “a patient” refers to a mammalian individual afflicted with or prone to a condition, disease or disorder as specified herein, and includes both humans and animals.

According to one embodiment of the invention, an adjustable neonatal airway stent has a large dilation range that can be tightly rolled for insertion into a small lumen, which can be successively expanded as the lumen grows. Moreover, the adjustable neonatal airway stent of the invention may facilitate epithilialization to anchor the stent, to enhance protection of the airway mucosal layer from denudation during mechanical ventilation, and/or to enable the cilia to function normally. The adjustable neonatal airway stent may support the airway against intermittent fluctuating negative and positive pressures and may be used to encourage expansion and growth of an underdeveloped airway.

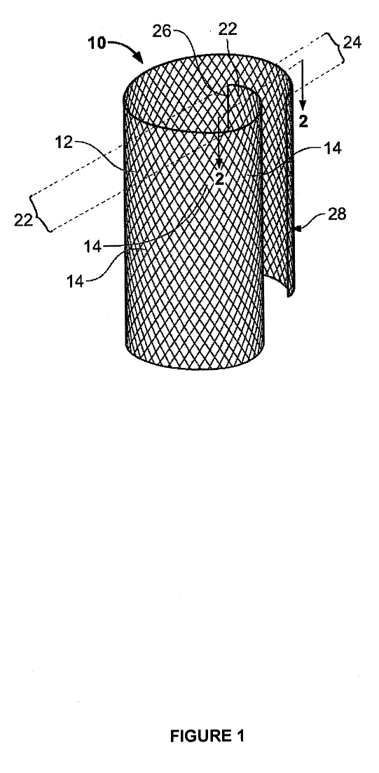

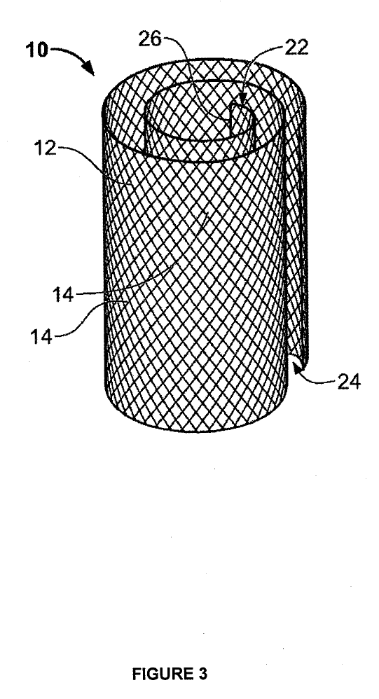

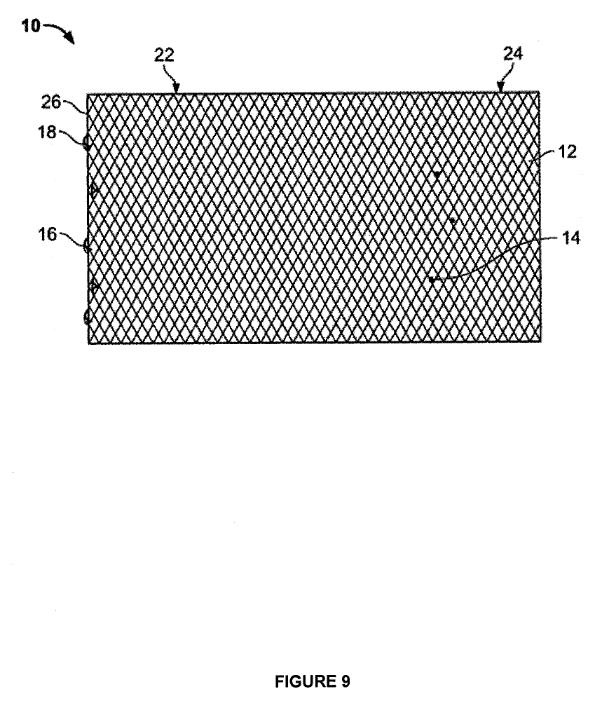

As illustrated in FIG. 1, one embodiment of a neonatal stent 10 of the invention includes a fine flexible mesh member 12 rolled into a cylindrical shape such that an inner edge portion 22 is overlapped by outer edge portion 24. Prior to insertion into a neonatal airway, the stent 10 is rolled as shown in FIG. 3 to a sufficiently small diameter to facilitate delivery into the airway of a neonate. The small size of a neonatal airway requires that the mesh material 12 of the stent 10 be capable of being rolled tightly into a cylinder while retaining its ability to unwind back into a nearly flat sheet. If unrolled to a completely flat configuration, such as shown in FIG. 9, the mesh material 12 would be generally rectangular in shape, noting that rectangular is meant to include square but other shapes could be used as well. The neonatal stent for use in primary airways may have an outside diameter in a range of about 3 mm to about 6 mm, and for use in secondary airways may have an outside diameter in a range of about 1.5 mm to about 3 mm.

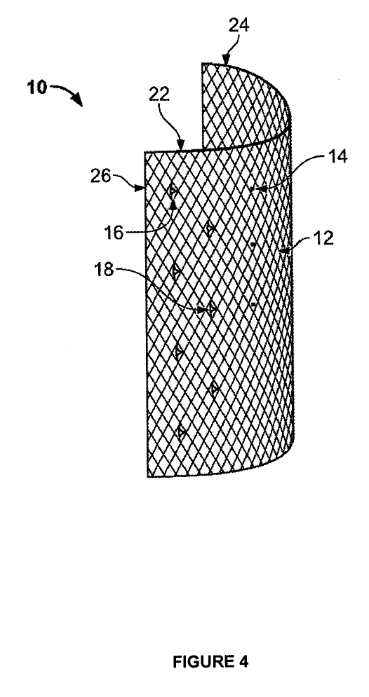

Subsequent to insertion into the airway, the stent 10 is expanded by any means known in the art, such as being made of a self-expanding material that expands either under force of its own spring action or recoil after being released from a delivery sheath, or by way of a balloon catheter inserted into the lumen of the stent, until it is in intimate contact with the inner wall of the trachea. An exemplary view of a pre-stressed mesh member 12 of the invention 10 is illustrated in FIG. 4. The mesh member 12 that rests naturally in the partially rolled up shape shown in FIG. 4 will be elastically motivated to revert towards that shape after being released within the trachea. The pressure employed within the tracheal balloon, or by the preset recoil, can be controlled to expand the stent 10 to the desired size, ensuring that sufficient but not excessive interactional force exists between the stent 10 and the airway wall.

As shown in FIG. 1, an inner axial edge 26 of the flexible mesh member 12 defines one circumferential boundary of the inner edge portion 22. When the flexible mesh member 12 is rolled up into a cylindrical shape so that inner edge portion 22 and outer edge portion 24 overlap, the inner axial edge 26 forms one boundary of the overlapping inner and outer portions 22, 24 of the mesh 12, and the outer axial edge 28 defines the other circumferential boundary.

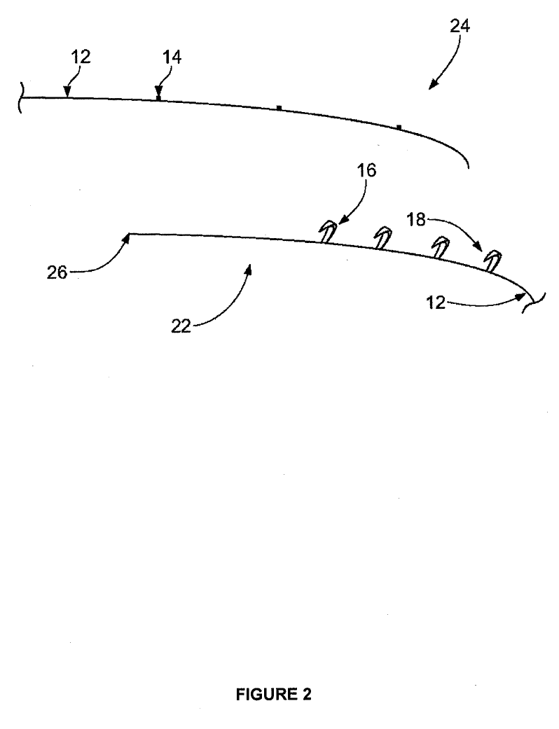

As seen best in FIGS. 2, 4, and 10, a plurality of ratcheting barbs 16 to protrude outwardly from the outer wall of the stent in or near inner edge portion 22. The barbs 16 are engagable with the overlapping outer portion 24 of the mesh 12.

As illustrated in FIG. 2, the barbs 16 may include backward extending tips 18. The barbs 16 are disposed at an acute angle with respect to the mesh inner edge portion 22, and each backward extending tip 18 is disposed at an acute angle with respect to its respective barb 16. The barbs 16 are additionally disposed at an angle at least partially towards the stent inner axial edge 26. The barbs 16 provide a one-way ratcheting feature such that the stent 10 can be expanded but is prevented from collapsing inwardly. The stent diameter remains constant until an appropriate force is applied to expand the stent. The angle between the barbs 16 and the inner edge portion 22 is preferably in the range from about ten degrees to about sixty degrees, but any angle between zero and ninety degrees may be used.

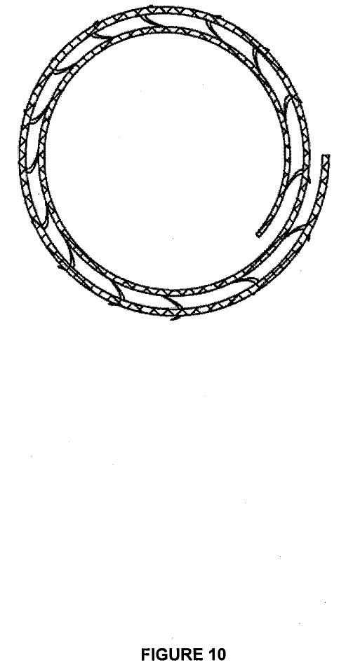

As shown best in FIG. 10, the backward extending tips 18 engage the mesh 12 of the overlapping outer edge portion 24 to provide an additional interconnection between the inner and outer edge portions 22, 24. The interaction between the mesh outer edge portion 24 and the tips 18 of the barbs 16 is sufficiently strong to preserve intimate contact between the inner and outer edge portions 22, 24, preventing separation between the respective outer and inner walls thereof, yet is resilient enough to allow the inner and outer edge portions 22, 24 to move parallel to one another as the stent 10 is expanded under either its own recoil or the force of a balloon catheter inserted thereinto. The ratcheting action of the barbs 16 themselves provides a sufficiently strong interactional force to prevent the stent 10 from contracting in diameter under the force of the walls of the lumen into which the stent 10 is inserted.

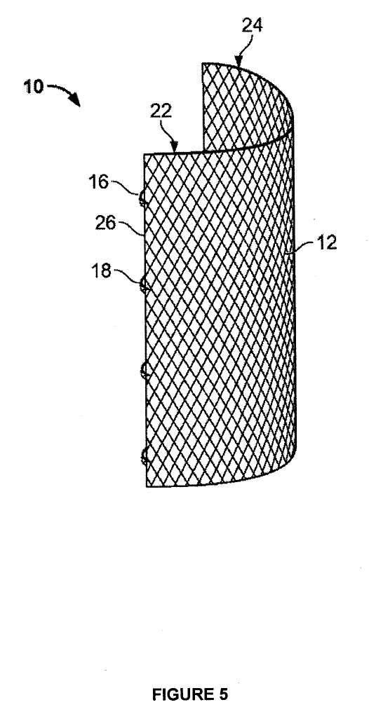

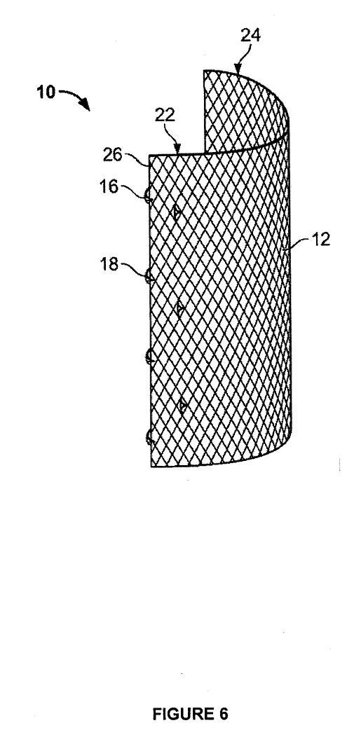

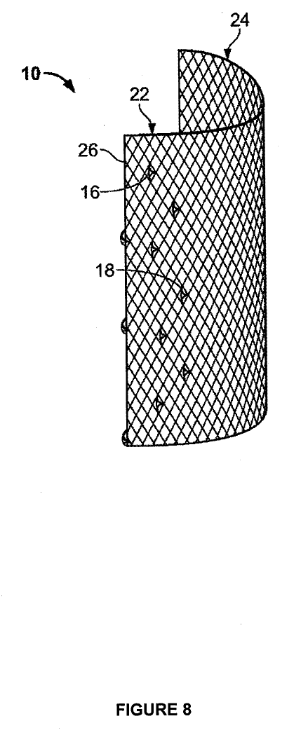

The barbs 16 may be disposed in one or more staggered rows running generally parallel to the inner axial edge 26 with at least one row being spaced from the inner edge 26, as show in FIG. 4. An alternate embodiment of the stent 10, shown in FIG. 5, has barbs 16 disposed in a single row only along the inner edge 26. A further alternate embodiment has at least one barb 16 disposed at the inner edge 26 and at least one barb 16 disposed at a distance from the inner edge 26, exemplified in FIG. 6. In yet another alternative embodiment, exemplified in FIG. 8, the barbs 16 may be placed in a patterned, irregular, or clustered array spread about the outer wall of the inner edge portion 22.

A barb 16 may be one of a variety of projections extending from the inner edge portion 22 and angling at least partially towards the inner axial edge 26 so as to engage the overlapping outer edge portion 24. Each barb 16 need not extend perpendicularly toward the inner axial edge 26, but may be angled as exemplified in FIG. 2. The term barbs are used in a broad sense and may be in the form of straight rigid spikes, semi-rigid spikes, curved spikes, straight teeth, pointed teeth, curved teeth, tabs or other projections capable of permitting limited, relative movement of the overlapping portions 22, 24 to allow expansion of the lumen of the stent 10 but prevent contraction thereof. The number and arrangement of the barbs 16 can vary, so long as at least one barb 16 is spaced from the inner axial edge 26. Barbs 16 disposed at a distance from the inner edge 26 provide an increased intimacy of contact between the inner and outer overlapped edge portions 22, 24 as compared with an arrangement having barbs or other projections disposed only at the inner edge 26. This more intimate contact between the inner and outer overlapping portions 22, 24 improves the structural stability of the stent 10 and enhances the ability of the stent 10 to be epithilialized in a neonatal trachea. In combination with the barbs 16 disposed at a distance from the inner edge 26, the backward extending tips 18 provide further enhanced intimacy of contact between the inner and outer overlapping portions 22, 24. Additionally, multiple rows of barbs 16, or a plurality of barbs 16 spaced at more than one distance from the inner axial edge 26, similarly increases the structural stability and epithilialization of the stent 10.

The mesh construction 12 of the stent 10 provides for epithilialization (mucosalization) of the stent 10, whereby the mucosal layer protrudes through the mesh 12 and becomes entwined therewith. As a result, mucus produced by the trachea walls reaches the airway and the cilia extend through the mesh 12 to function unimpeded. Epithilialization additionally decreases the possibility of migration of the stent 10 along the airway. Migration can be further minimized by incorporating studs 14, which may be smaller projections than the barbs 16 disposed on the outer wall of the stent 10, as illustrated in FIG. 2, to facilitate anchoring to impede longitudinal movement along the axis of the airway.

The flexible mesh 12 and barbs 16 are preferably constructed from biocompatible hypoallergenic materials. Such biocompatible resilient materials may include stainless steel, titanium, platinum, tantalum, or tungsten alloy. A particular material may be a thermal shape-memory polymer or metal, or a super-elastic material such as nitinol (a nickel-titanium alloy). Various plastics or polymers may also be used, including those with microporous structures such as silicone, polyurethane, poly vinyl alcohol, polyethylene, polyesters, hydrogels, tetrafluoroethylene, fuorosilicone, hyaluronte, and combinations, copolymers, and blended mixtures thereof. Additionally, bioabsorbable plastics such as biodegradable poly-L-lactide, polylactic acid, polygloycolic acid, polycaprolactone, or other members of the linear aliphatic polyester family or associated copolymers, may be used. Other bioabsorbable materials suitable for the invention may include poly-D, L-lactic acid, polyethylene glycol, polydioxanone, polylactic acid, 70L/30DL polylactide, polyglycolide, poly(orthoester), calcium sodium metaphosphate, hydroxyapatite, calcium phosphate, polytetra fluoroethylene, collagen I, II, IX, X, and XI, durapatite, and hydrogel.

Nitinol is particularly attractive for applications were a self-expanding stent is desired, since it is a superelastic nickel-titanium alloy with a distinct shape memory. The preferred nitinol has an austenite transition temperature slightly below body temperature, so that the rolled mesh exhibits superelastic behavior when deployed. This retained shape memory can be altered by the application of heat. Therefore, a nitinol stent may be formed to a particular shape, coiled tightly for insertion, and then released at the desired intraluminal location in the airway where it will strive to return to its preset shape.

Poly-L-lactide, a bioabsorptive material, is particularly attractive for applications where a stent is anticipated to be needed only for a limited amount of time and it is desired to avoid the step of having to remove the stent once its function is complete. A poly-L-lactide stent is completely bioabsorbable, and the duration of its effectiveness as a stent can be manipulated by varying the basic molecular construction of the poly-L-lactide. Additionally, poly-L-lactide stents are tolerated as well as metallic stents and better than those made from silicone.

The diameter of the stent 10 may be adjustable, thereby making the stent dynamically adaptable to growth. As the airway enlarges during growth and development of the trachea, the stent 10 can be expanded to match the changed size of the airway. Typically, this will be done after an angiogram or other procedure in which a physician can determine the size of the airway. Because the stent 10 may be formed from a mesh that is loosely rolled in its inherent or unstressed state, the natural elasticity of the stent 10 itself will provide some ability for simultaneous and coordinated growth of the stent 10 within the growing trachea. In addition, the stent 10 may be expanded periodically by the intermittent use of a tracheal balloon. In either case, the one-way ratcheting barbs 16 allow the stent 10 to expand but not to contract.

Once in place, the stent 10 provides a cylindrical shape and elastic structural support that decreases the compliance of the airway under serial pressure fluctuations. The stent 10 applies a dispersed and uniform outward pressure to support the airway, thereby resisting collapse that could otherwise occur under negative pressure spontaneous or forced inspiratory efforts. Contraction of the stent 10 is mechanically prevented by the engagement of the ratcheting barbs 16 with the mesh 12. The structural rigidity of the stent 10 also aids the trachea in resisting deformation during positive pressure expiratory efforts, either spontaneous or mechanical. The mucosalization of the stent 10 within the airway enhances the ability of the stent 10 to provide mechanical support to the trachea under both inspiratory and expiratory conditions.

Further, the stent 10 may be used to apply a constant outward distending pressure as a stimulus for growth and expansion of the hypoplastic airway. Towards this end, the stent 10 can be repeatedly and successively expanded over time, e.g., by using a tracheal balloon, to encourage enlargement of an airway suffering from retarded growth.

The ability of the stent 10 to expand, either in response to or in encouragement of airway growth, ensures that the stent 10 will not become an obstruction as the airway grows. Depending on its material of construction, the stent 10 may be removed following maturation of the airway, or it may remain in place through adulthood as a relatively small piece of mesh embedded in one wall of the larger trachea.

The mesh structure 12 of the stent 10 protects the mucosal lining of the airway from denudation during mechanical ventilation. The mesh 12 is fine enough to permit epithialization without sacrificing structural strength. In-growth of the mucosal layer around the fibers of the mesh 12 helps to stabilize the mucosal lining and increases its resistance to damage by the forced air flow and intermittent pressure fluctuations induced by mechanical ventilation.

According to another feature of the invention, growth and/or healing may be induced during the surgical procedure of the invention by introducing growth factors and/or stem cells to the target area undergoing correction. The growth factors and/or stem cells could be delivered to the target area by a variety of methods. One method of delivery may be to coat the stent with the growth factor in combination with hydroxyapatite. For this to be accomplished, an effective amount of the bone growth factor would be absorbed to a gritblasted hydroxyapatite coated stent prior to implantation into the patient.

However, an alternate method to the delivery of recombinant growth factors may be through gene therapy. Delivery by gene therapy may be more cost effective because ex vivo production of DNA for clinical use is inexpensive compared with traditional methods of protein production. Also, gene therapy may be a more efficient way to deliver the growth factors compared with traditional protein delivery. One desirable way to utilize gene therapy in the surgical procedure of the invention may be to introduce plasmid-encoded proteins capable of inducing cell growth to the target area. This may be accomplished by introducing biodegradable matrices, such as collagen sponges, containing expression plasmid DNA encoding growth factors, also known as gene-activated matrices (GAMs), to the target area.

In an additional embodiment, the mesh 12 and barbs 16 may be treated with bioactive agents to influence tissue growth and to facilitate epithilialization, further enabling a viable and stable mucosal lining of the airway. Such bioactive agents may include heparin, prostacyclenes and analogs thereof, antithrombogenic agents, steroids, ibuprofen, antimicrobials, antibiotics, tissue plasma activators, rifamicin, monoclonal antibodies, snake venom protein by-products, antifibrosis agents, cyclosporine, hyaluronte, and mixtures of these bioactive substances for simultaneous multiple treatments. It is recognized that virtually any bioactive agent of need to the neonate is a possible agent that could be applied using the stent of the invention. Bioactive agents include the following categories and specific examples of the various agents. It is not intended that the category be limited by the specific examples. Those of ordinary skill in the art will recognize numerous other compounds that fall within the categories and that are useful for promoting growth and/or healing of the target area, such as the particular airway undergoing treatment.

Adrenocortical steroid: Ciprocinonide; Desoxycorticosterone Acetate; Desoxycorticosterone Pivalate; Dexamethasone Acetate; Fludrocortisone Acetate; Flumoxonide; Hydrocortisone Hemisuccinate; Methylprednisolone Hemisuccinate; Naflocort; Procinonide; Timobesone Acetate; Tipredane.

Adrenocortical suppressant: Aminoglutethimide; and Trilostane.

Analgesic: Acetaminophen; Alfentanil Hydrochloride; Aminobenzoate Potassium; Aminobenzoate Sodium; Anidoxime; Anileridine; Anileridine Hydrochloride; Anilopam Hydrochloride; Anirolac; Antipyrine; Aspirin; Benoxaprofen; Benzydamine Hydrochloride; Bicifadine Hydrochloride; Brifentanil Hydrochloride; Bromadoline Maleate; Bromfenac Sodium; Buprenorphine Hydrochloride; Butacetin; Butixirate; Butorphanol; Butorphanol Tartrate; Carbamazepine; Carbaspirin Calcium; Carbiphene Hydrochloride; Carfentanil Citrate; Ciprefadol Succinate; Ciramadol; Ciramadol Hydrochloride; Clonixeril; Clonixin; Codeine; Codeine Phosphate; Codeine Sulfate; Conorphone Hydrochloride; Cyclazocine; Dexoxadrol Hydrochloride; Dexpemedolac; Dezocine; Diflunisal; Dihydrocodeine Bitartrate; Dimefadane; Dipyrone; Doxpicomine Hydrochloride; Drinidene; Enadoline Hydrochloride; Epirizole; Ergotamine Tartrate; Ethoxazene Hydrochloride; Etofenamate; Eugenol; Fenoprofen; Fenoprofen Calcium; Fentanyl Citrate; Floctafenine; Flufenisal; Flunixin; Flunixin Meglumine; Flupirtine Maleate; Fluproquazone; Fluradoline Hydrochloride; Flurbiprofen; Hydromorphone Hydrochloride; Ibufenac; Indoprofen; Ketazocine; Ketorfanol; Ketorolac Tromethamine; Letimide Hydrochloride; Levomethadyl Acetate; Levomethadyl Acetate Hydrochloride; Levonantradol Hydrochloride; Levorphanol Tartrate; Lofemizole Hydrochloride; Lofentanil Oxalate; Lorcinadol; Lomoxicam; Magnesium Salicylate; Mefenamic Acid; Menabitan Hydrochloride; Meperidine Hydrochloride; Meptazinol Hydrochloride; Methadone Hydrochloride; Methadyl Acetate; Methopholine; Methotrimeprazine; Metkephamid Acetate; Mimbane Hydrochloride; Mirfentanil Hydrochloride; Molinazone; Morphine Sulfate; Moxazocine; Nabitan Hydrochloride; Nalbuphine Hydrochloride; Nalmexone Hydrochloride; Namoxyrate; Nantradol Hydrochloride; Naproxen; Naproxen Sodium; Naproxol; Nefopam Hydrochloride; Nexeridine Hydrochloride; Noracymethadol Hydrochloride; Ocfentanil Hydrochloride; Octazamide; Olvanil; Oxetorone Fumarate; Oxycodone; Oxycodone Hydrochloride; Oxycodone Terephthalate; Oxymorphone Hydrochloride; Pemedolac; Pentamorphone; Pentazocine; Pentazocine Hydrochloride; Pentazocine Lactate; Phenazopyridine Hydrochloride; Phenyramidol Hydrochloride; Picenadol Hydrochloride; Pinadoline; Pirfenidone; Piroxicam Olamine; Pravadoline Maleate; Prodilidine Hydrochloride; Profadol Hydrochloride; Propirarn Fumarate; Propoxyphene Hydrochloride; Propoxyphene Napsylate; Proxazole; Proxazole Citrate; Proxorphan Tartrate; Pyrroliphene Hydrochloride; Remifentanil Hydrochloride; Salcolex; Salethamide Maleate; Salicylamide; Salicylate Meglumine; Salsalate; Sodium Salicylate; Spiradoline Mesylate; Sufentanil; Sufentanil Citrate; Talmetacin; Talniflumate; Talosalate; Tazadolene Succinate; Tebufelone; Tetrydamine; Tifurac Sodium; Tilidine Hydrochloride; Tiopinac; Tonazocine Mesylate; Tramadol Hydrochloride; Trefentanil Hydrochloride; Trolamine; Veradoline Hydrochloride; Verilopam Hydrochloride; Volazocine; Xorphanol Mesylate; Xylazine Hydrochloride; Zenazocine Mesylate; Zomepirac Sodium; and Zucapsaicin.

Anti-asthmatic: Ablukast; Ablukast Sodium; Azelastine Hydrochloride; Bunaprolast; Cinalukast; Cromitrile Sodium; Cromolyn Sodium; Enofelast; Isamoxole; Ketotifen Fumarate; Levcromakalim; Lodoxamide Ethyl; Lodoxamide Tromethamine; Montelukast Sodium; Ontazolast; Oxarbazole; Oxatomide; Piriprost; Piriprost Potassium; Pirolate; Pobilukast Edamine; Quazolast; Repirinast; Ritolukast; Sulukast; Tetrazolast Meglumine; Tiaramide Hydrochloride; Tibenelast Sodium; Tomelukast; Tranilast; Verlukast; Verofylline; Zarirlukast. Antibacterial: Acedapsone; Acetosulfone Sodium; Alamecin; Alexidine; Amdinocillin, Amdinocillin Pivoxil; Amicycline; Amifloxacin; Amifloxacin Mesylate; Amikacin; Amikacin Sulfate; Aminosalicylic acid; Aminosalicylate sodium; Amoxicillin; Amphomycin; Ampicillin; Ampicillin Sodium; Apalcillin Sodium; Apramycin; Aspartocin; Astromicin Sulfate; Avilamycin; Avoparcin; Azithromycin; Azlocillin; Azlocillin Sodium; Bacampicillin Hydrochloride; Bacitracin; Bacitracin Methylene Disalicylate; Bacitracin Zinc; Bambermycins; Benzoylpas Calcium; Berythromycin; Betamicin Sulfate; Biapenem; Biniramycin; Biphenamine Hydrochloride; Bispyrithione Magsulfex; Butikacin; Butirosin Sulfate; Capreomycin Sulfate; Carbadox; Carbenicillin Disodium; Carbenicillin Indanyl Sodium; Carbenicillin Phenyl Sodium; Carbenicillin Potassium; Carumonam Sodium; Cefaclor; Cefadroxil; Cefamandole; Cefamandole Nafate; Cefamandole Sodium; Cefaparole; Cefatrizine; Cefazaflur Sodium; Cefazolin; Cefazolin Sodium; Cefbuperazone; Cefdinir; Cefepime; Cefepime Hydrochloride; Cefetecol; Cefixime; Cefmenoxime Hydrochloride; Cefmetazole; Cefmetazole Sodium; Cefonicid Monosodium; Cefonicid Sodium; Cefoperazone Sodium; Ceforanide; Cefotaxime Sodium; Cefotetan; Cefotetan Disodium; Cefotiam Hydrochloride; Cefoxitin; Cefoxitin Sodium; Cefpimizole; Cefpimizole Sodium; Cefpiramide; Cefpiramide Sodium; Cefpirome Sulfate; Cefpodoxime Proxetil; Cefprozil; Cefroxadine; Cefsulodin Sodium; Ceftazidime; Ceftibuten; and Ceftizoxime.

Antifungal: Acrisorcin; Ambruticin; Amphotericin B; Azaconazole; Azaserine; Basifungin; Bifonazole; Biphenamine Hydrochloride; Bispyrithione Magsulfex; Butoconazole Nitrate; Calcium Undecylenate; Candicidin; Carbol-Fuchsin; Chlordantoin; Ciclopirox; Ciclopirox Olamine; Cilofungin; Cisconazole; Clotrimazole; Cuprimyxin; Denofungin; Dipyrithione; Doconazole; Econazole; Econazole Nitrate; Enilconazole; Ethonam Nitrate; Fenticonazole Nitrate; Filipin; Fluconazole; Flucytosine; Fungimycin; Griseofulvin; Hamycin; Isoconazole; Itraconazole; Kalafungin; Ketoconazole; Lomofimgin; Lydimycin; Mepartricin; Miconazole; Miconazole Nitrate; Monensin; Monensin Sodium; Naftifine Hydrochloride; Neomycin Undecylenate; Nifuratel; Nifurmerone; Nitralamine Hydrochloride; Nystatin; Octanoic Acid; Orconazole Nitrate; Oxiconazole Nitrate; Oxifungin Hydrochloride; Parconazole Hydrochloride; Partricin; Potassium Iodide; Proclonol; Pyrithione Zinc; Pyrrolnitrin; Rutamycin; Sanguinarium Chloride; Saperconazole; Scopafungin; Selenium Sulfide; Sinefungin; Sulconazole Nitrate; Terbinafine; Terconazole; Thiram; Ticlatone; Tioconazole; Tolciclate; Tolindate; Tolnaftate; Triacetin; Triafungin; Undecylenic Acid; Viridofulvin; and Zinc Undecylenate; Zinoconazole Hydrochloride.

Antihistaminic: Acrivastine; Antazoline Phosphate; Astemizole; Azatadine Maleate; Barmastine; Bromodiphenhydramine Hydrochloride; Brompheniramnine Maleate; Carbinoxamine Maleate; Cetirizine Hydrochloride; Chlorpheniramine Maleate; Chlorpheniramine Polistirex; Cinnarizine; Clemastine; Clemastine Fumarate; Closiramine Aceturate; Cycliramine Maleate; Cyclizine; Cyproheptadine Hydrochloride; Dexbrompheniramnine Maleate; Dexchlorpheniramine Maleate; Dimethindene Maleate; Diphenhydramine Citrate; Diphenhydramnine Hydrochloride; Dorastine Hydrochloride; Doxylamine Succinate; Ebastine; Levocabastine Hydrochloride; Loratadine; Mianserin Hydrochloride; Noberastine; Orphenadrine Citrate; Pyrabrom; Pyrilamine Maleate; Pyroxamnine Maleate; Rocastine Hydrochloride; Rotoxamine; Tazifylline Hydrochloride; Temelastine; Terfenadine; Tripelennamine Citrate; Tripelennamine Hydrochloride; Triprolidine Hydrochloride; and Zolamine Hydrochloride.

Anti-infective: Difloxacin Hydrochloride; Lauryl Isoquinolinium Bromide; Moxalactam Disodium; Omidazole; Pentisomicin; Sarafloxacin Hydrochloride; Protease inhibitors of HIV and other retroviruses; Integrase Inhibitors of HIV and other retroviruses; Cefaclor (Ceclor); Acyclovir (Zovirax); Norfloxacin (Noroxin); Cefoxitin (Mefoxin); Cefuroxime axetil (Ceftin); and Ciprofloxacin (Cipro).

Anti-inflammatory: Alclofenac; Alclometasone Dipropionate; Algestone Acetonide; Alpha Amylase; Amcinafal; Amcinafide; Amfenac Sodium; Amiprilose Hydrochloride; Anakinra; Anirolac; Anitrazafen; Apazone; Balsalazide Disodium; Bendazac; Benoxaprofen; Benzydamine Hydrochloride; Bromelains; Broperamole; Budesonide; Carprofen; Cicloprofen; Cintazone; Cliprofen; Clobetasol Propionate; Clobetasone Butyrate; Clopirac; Cloticasone Propionate; Cormethasone Acetate; Cortodoxone; Deflazacort; Desonide; Desoximetasone; Dexamethasone Dipropionate; Diclofenac Potassium; Diclofenac Sodium; Diflorasone Diacetate; Diflumidone Sodium; Diflunisal; Difluprednate; Diftalone; Dimethyl Sulfoxide; Drocinonide; Endrysone; Enlimomab; Enolicam Sodium; Epirizole; Etodolac; Etofenamate; Felbinac; Fenamole; Fenbufen; Fenclofenac; Fenclorac; Fendosal; Fenpipalone; Fentiazac; Flazalone; Fluazacort; Flufenamic Acid; Flumizole; Flunisolide Acetate; Flunixin; Flunixin Meglumine; Fluocortin Butyl; Fluorometholone Acetate; Fluquazone; Flurbiprofen; Fluretofen; Fluticasone Propionate; Furaprofen; Furobufen; Halcinonide; Halobetasol Propionate; Halopredone Acetate; Ibufenac; Ibuprofen; Ibuprofen Aluminum; Ibuprofen Piconol; Ilonidap; Indomethacin; Indomethacin Sodium; Indoprofen; Indoxole; Intrazole; Isoflupredone Acetate; Isoxepac; Isoxicam; Ketoprofen; Lofemizole Hydrochloride; Lomoxicam; Loteprednol Etabonate; Meclofenamate Sodium; Meclofenamic Acid; Meclorisone Dibutyrate; Mefenamic Acid; Mesalamine; Meseclazone; Methylprednisolone Suleptanate; Momiflumate; Nabumetone; Naproxen; Naproxen Sodium; Naproxol; Nimazone; Olsalazine Sodium; Orgotein; Orpanoxin; Oxaprozin; Oxyphenbutazone; Paranyline Hydrochloride; Pentosan Polysulfate Sodium; Phenbutazone Sodium Glycerate; Pirfenidone; Piroxicam; Piroxicam Cinnamate; Piroxicam Olamine; Pirprofen; Prednazate; Prifelone; Prodolic Acid; Proquazone; Proxazole; Proxazole Citrate; Rimexolone; Romazarit; Salcolex; Salnacedin; Salsalate; Sanguinarium Chloride; Seclazone; Sermetacin; Sudoxicam; Sulindac; Suprofen; Talmetacin; Talniflumate; and Talosalate.

Antineoplastic: Acivicin; Aclarubicin; Acodazole Hydrochloride; AcrQnine; Adozelesin; Aldesleukin; Altretamine; Ambomycin; Ametantrone Acetate; Aminoglutethimide; Amsacrine; Anastrozole; Anthramycin; Asparaginase; Asperlin; Azacitidine; Azetepa; Azotomycin; Batimastat; Benzodepa; Bicalutamide; Bisantrene Hydrochloride; Bisnafide Dimesylate; Bizelesin; Bleomycin Sulfate; Brequinar Sodium; Bropirimine; Busulfan; Cactinomycin; Calusterone; Caracemide; Carbetimer; Carboplatin; Carmustine; Carubicin Hydrochloride; Carzelesin; Cedefingol; Chlorambucil; Cirolemycin; Cisplatin; Cladribine; Crisnatol Mesylate; Cyclophosphamide; Cytarabine; Dacarbazine; Dactinomycin; Daunorubicin Hydrochloride; Decitabine; Dexormaplatin; Dezaguanine; Dezaguanine Mesylate; Diaziquone; Docetaxel; Doxorubicin; Doxorubicin Hydrochloride; Droloxifene; Droloxifene Citrate; Dromostanolone Propionate; Duazomycin; Edatrexate; Eflomithine Hydrochloride; Elsamitrucin; Enloplatin; Enpromate; Epipropidine; Epirubicin Hydrochloride; Erbulozole; Esorubicin Hydrochloride; Estramustine; Estramustine Phosphate Sodium; Etanidazole; Ethiodized Oil I 131; Etoposide; Etoposide Phosphate; Etoprine; Fadrozole Hydrochloride; Fazarabine; Fenretinide; Floxuridine; Fludarabine Phosphate; Fluorouracil; Flurocitabine; Fosquidone; Fostriecin Sodium; Gemcitabine; Gemcitabine Hydrochloride; Gold Au 198; Hydroxyurea; Idarubicin Hydrochloride; Ifosfamide; Ilmofosine; Interferon Alfa-2a; Interferon Alfa-2b; Interferon Alfa-n1; Interferon Alfa-n3; Interferon Beta-Ia; Interferon Gamma-Ib; Iproplatin; Irinotecan Hydrochloride; Lanreotide Acetate; Letrozole; Leuprolide Acetate; Liarozole Hydrochloride; Lometrexol Sodium; Lomustine; Losoxantrone Hydrochloride; Masoprocol; Maytansine; Mechlorethamine Hydrochloride; Megestrol Acetate; Melengestrol Acetate; Melphalan; Menogaril; Mercaptopurine; Methotrexate; Methotrexate Sodium; Metoprine; Meturedepa; Mitindomide; Mitocarcin; Mitocromin; Mitogillin; Mitomalcin; Mitomycin; Mitosper; Mitotane; Mitoxantrone Hydrochloride; Mycophenolic Acid; Nocodazole; Nogalamycin; Ormaplatin; Oxisuran; Paclitaxel; Pegaspargase; Peliomycin; Pentamustine; Peplomycin Sulfate; Perfosfamide; Pipobroman; Piposulfan; Piroxantrone Hydrochloride; Plicamycin; Plomestane; Porfimer Sodium; Porfiromycin; Prednimustine; Procarbazine Hydrochloride; Puromycin; Puromycin Hydrochloride; Pyrazofurin; Riboprine; Rogletimide; Safmgol; Safingol Hydrochloride; Semustine; Simtrazene; Sparfosate Sodium; Sparsomycin; Spirogermanium Hydrochloride; Spiromustine; Spiroplatin; Streptonigrin; Streptozocin; Strontium Chloride Sr 89; Sulofenur; Talisomycin; Taxane; Taxoid; Tecogalan Sodium; Tegafur; Teloxantrone Hydrochloride; Temoporfin; Teniposide; Teroxirone; Testolactone; Thiamiprine; Thioguanine; Thiotepa; Tiazofurin; Tirapazamine; Topotecan Hydrochloride; Toremifene Citrate; Trestolone Acetate; Triciribine Phosphate; Trimetrexate; Trimetrexate Glucuronate; Triptorelin; Tubulozole Hydrochloride; Uracil Mustard; Uredepa; Vapreotide; Verteporfin; Vinblastine Sulfate; Vincristine Sulfate; Vindesine; Vindesine Sulfate; Vinepidine Sulfate; Vinglycinate Sulfate; Vinleurosine Sulfate; Vinorelbine Tartrate; Vinrosidine Sulfate; Vinzolidine Sulfate; Vorozole; Zeniplatin; Zinostatin; and Zorubicin Hydrochloride.

Antiviral: Acemannan; Acyclovir; Acyclovir Sodium; Adefovir; Alovudine; Alvircept Sudotox; Amantadine Hydrochloride; Aranotin; Arildone; Atevirdine Mesylate; Avridine; Cidofovir; Cipamfylline; Cytarabine Hydrochloride; Delavirdine Mesylate; Desciclovir; Didanosine; Disoxaril; Edoxudine; Enviradene; Enviroxime; Famciclovir; Famotine Hydrochloride; Fiacitabine; Fialuridine; Fosarilate; Foscamet Sodium; Fosfonet Sodium; Ganciclovir; Ganciclovir Sodium; Idoxuridine; Kethoxal; Lamivudine; Lobucavir; Memotine Hydrochloride; Methisazone; Nevirapine; Penciclovir; Pirodavir; Ribavirin; Rimantadine Hydrochloride; Saquinavir Mesylate; Somantadine Hydrochloride; Sorivudine; Statolon; Stavudine; Tilorone Hydrochloride; Trifluridine; Valacyclovir Hydrochloride; Vidarabine; Vidarabine Phosphate; Vidarabine Sodium Phosphate; Viroxime; Zalcitabine; Zidovudine; and Zinviroxime.

Bronchodilator: Albuterol; Albuterol Sulfate; Azanator Maleate; Bamifylline Hydrochloride; Bitolterol Mesylate; Butaprost; Carbuterol Hydrochloride; Clorprenaline Hydrochloride; Colterol Mesylate; Doxaprost; Doxofylline; Dyphylline; Enprofylline; Ephedrine; Ephedrine Hydrochloride; Fenoterol; Fenprinast Hydrochloride; Guaithylline; Hexoprenaline Sulfate; Hoquizil Hydrochloride; Ipratropium Bromide; Isoetharine; Isoetharine Hydrochloride; Isoetharine Mesylate; Isoproterenol Hydrochloride; Isoproterenol Sulfate; Metaproterenol Polistirex; Metaproterenol Sulfate; Nisbuterol Mesylate; Oxtriphylline; Picumeterol Fumarate; Piquizil Hydrochloride; Pirbuterol Acetate; Pirbuterol Hydrochloride; Procaterol Hydrochloride; Pseudoephedrine Sulfate; Quazodine; Quinterenol Sulfate; Racepinephrine; Racepinephrine Hydrochloride; Reproterol Hydrochloride; Rimiterol Hydrobromide; Salmeterol; Salmeterol Xinafoate; Soterenol Hydrochloride; Sulfonterol Hydrochloride; Suloxifen Oxalate; Terbutaline Sulfate; Theophylline; Xanoxate Sodium; Zindotrine; and Zinterol Hydrochloride.

Glucocorticoid: Amcinonide; Beclomethasone Dipropionate; Betamethasone; Betamethasone Acetate; Betamethasone Benzoate; Betamethasone Dipropionate; Betamethasone Sodium Phosphate; Betamethasone Valerate; Carbenoxolone Sodium; Clocortolone Acetate; Clocortolone Pivalate; Cloprednol; Corticotropin; Corticotropin, Repository; Corticotropin Zinc Hydroxide; Cortisone Acetate; Cortivazol; Descinolone Acetonide; Dexamethasone; Dexamethasone Sodium Phosphate; Diflucortolone; Diflucortolone Pivalate; Flucloronide; Flumethasone; Flumethasone Pivalate; Flunisolide; Fluocinolone Acetonide; Fluocinonide; Fluocortolone; Fluocortolone Caproate; Fluorometholone; Fluperolone Acetate; Fluprednisolone; Fluprednisolone Valerate; Flurandrenolide; Formocortal; Hydrocortisone; Hydrocortisone Acetate; Hydrocortisone Buteprate; Hydrocortisone Butyrate; Hydrocortisone Sodium Phosphate; Hydrocortisone Sodium Succinate; Hydrocortisone Valerate; Medrysone; Methylprednisolone; Methylprednisolone Acetate; Methylprednisoloime Sodium Phosphate; Methylprednisolone Sodium Succinate; Nivazol; Paramethasone Acetate; Prednicarbate; Prednisolone; Prednisolone Acetate; Prednisolone Hemisuccinate; Prednisolone Sodium Phosphate; Prednisolone Sodium Succinate; Prednisolone Tebutate; Prednisone; Prednival; Ticabesone Propionate; Tralonide; Triamcinolone; Triamcinolone Acetonide; Triamcinolone Acetonide Sodium; Triamcinolone Diacetate; and Triamcinolone Hexacetonide.

Vasoconstrictor: Angiotensin Amide; Felypressin; Methysergide; and Methysergide Maleate.

Vasodilator: Alprostadil; Azaclorzine Hydrochloride; Bamethan Sulfate; Bepridil Hydrochloride; Buterizine; Cetiedil Citrate; Chromonar Hydrochloride; Clonitrate; Diltiazem Hydrochloride; Dipyridamole; Droprenilamine; Erythrityl Tetranitrate; Felodipine; Flunarizine Hydrochloride; Fostedil; Hexobendine; Inositol Niacinate; Iproxamine Hydrochloride; Isosorbide Dinitrate; Isosorbide Mononitrate; Isoxsuprine Hydrochloride; Lidoflazine; Mefenidil; Mefenidil Fumarate; Mibefradil Dihydrochloride; Mioflazine Hydrochloride; Mixidine; Nafronyl Oxalate; Nicardipine Hydrochloride; Nicergoline; Nicorandil; Nicotinyl Alcohol; Nifedipine; Nimodipine; Nisoldipine; Oxfenicine; Oxprenolol Hydrochloride; Pentaerythritol Tetranitrate; Pentoxifylline; Pentrinitrol; Perhexiline Maleate; Pindolol; Pirsidomine; Prenylamine; Propatyl Nitrate; Suloctidil; Terodiline Hydrochloride; Tipropidil Hydrochloride; Tolazoline Hydrochloride; and Xanthinol Niacinate.

The description given above is merely illustrative and is not meant to be an exhaustive list of all possible embodiments, applications or modifications of the invention. Thus, various modifications and variations of the described methods and systems of the invention will be apparent to those skilled in the art without departing from the scope and spirit of the invention. Although the invention has been described in connection with specific embodiments, it should be understood that the invention as claimed should not be unduly limited to such specific embodiments. Indeed, various modifications of the described modes for carrying out the invention which are obvious to those skilled in the cellular and molecular biology fields, medical device field or related fields are intended to be within the scope of the appended claims.

The disclosures of all references and publications cited above are expressly incorporated by reference in their entireties to the same extent as if each were incorporated by reference individually.

What we claim is:

1. An adjustable neonatal airway stent comprising:
a flexible mesh member having a generally cylindrical shape and size capable of being delivered to a neonatal airway, said mesh member including overlapping inner and outer portions, said inner portion being adjacent to an inner edge of said flexible mesh;
a plurality of locking projections extending outwardly from said inner portion, said projections engaging said overlapping outer portion to prevent contraction of the stent but allow expansion of the stent to at least one first diameter when implanted in a neonatal airway, and permit the stent to expand in situ to a second diameter greater than the first diameter.

2. The stent of claim 1, wherein said locking projections comprise angularly extending tips.

3. The stent of claim 1, wherein at least one of said locking projections is disposed on said mesh inner portion at a distance from said mesh inner edge.

4. The stent of claim 1, wherein said flexible mesh member and said locking projections comprise biocompatible or hypoallergenic metal or plastic.

5. The stent assembly of claim 1, wherein said flexible mesh member and said locking projections are composed of a biabsorbable material.

6. The stent of claim 1, wherein said locking projections are disposed in at least one row generally parallel to said mesh inner edge.

7. The stent of claim 1, wherein said locking projections are disposed in at least two rows generally parallel to said mesh inner edge.

8. The stent of claim 1, wherein said locking projections are disposed in an irregular array on said overlapping mesh inner portion.

9. The stent of claim 1, wherein said locking projections are disposed along said mesh inner edge.

10. The stent of claim 1, wherein at least of said flexible mesh member and said locking projection is coated with a bioactive agent to be delivered to the airway of a neonate.

11. The stent of claim 10, wherein said bioactive agent is one or more compounds selected from the group consisting of an adrenocortical steroid, an adrenocortical suppressant, an analgesic, an anti-asthmatic, an antifungal, an antihistaminic, an anti-infective, an anti-inflammatory,an antineoplastic, an antiviral, a bronchodilator, a glucocorticoid, a vasoconstrictor, a vasodilator, a growth factor, and stem cells.

12. The stent of claim 1, wherein said stent has a diameter in the range of about 1.5 mm to about 6 mm.

13. The stent of claim 1, wherein said locking projections are selected from the group consisting of one-way locking projections, two-way locking projections, barbs, teeth, tabs, and fingers.

14. A method of protecting the mucosal layer of an airway in a neonate from denudation due to mechanical ventilation, said method comprising the steps of:
providing an adjustable diameter stent including a flexible mesh member having a plurality of locking projections extending outwardly from the mesh portion and being disposed about a portion of one side of the mesh member adjacent to an edge thereof;
rolling the mesh member into a collapsed position having a generally cylindrical shape and size with overlapping inner and outer portions such that the mesh is capable of fitting within a neonatal airway;
retaining the mesh member in the collapsed position;
delivering the stent into the airway of the neonate;
positioning the stent where desired in the airway; and
implanting the stent in an expanded position in contact with the airway so that the locking projections engage the mesh of the overlapping mesh outer portion to retain the stent in the expanded state.

15. The method of claim 14, further comprising the steps of:
inserting a balloon catheter within the cylindrically shaped mesh member;
pressurizing the balloon catheter to expand the stent to a further expanded state so that the locking projections engage another section of the overlapping mesh outer portion to retain the stent in the further expanded state; and
depressurizing and removing the balloon catheter from the airway.

16. The method of claim 14, wherein at least one of the locking projections is disposed on the mesh portion at a distance from the edge.

17. The method of claim 14, wherein the locking projections are disposed in at least one row on the overlapping mesh inner portion generally parallel to the mesh member edge when the mesh member is rolled up.

18. The method of claim 14, wherein the locking projections are disposed in an array on the overlapping mesh inner portion when the mesh member is rolled up.

19. The method of claim 14, wherein the locking projections are disposed along the mesh member edge.

20. A method of enhancing growth of an underdeveloped airway in a neonate, said method comprising the steps of:
providing an adjustable diameter stent including a flexible mesh member having a plurality of locking projections extending outwardly from the mesh portion and being disposed about a portion of one side of the mesh member adjacent to an edge thereof, the locking projections;
rolling the mesh member into a collapsed position having a generally cylindrical shape and size with overlapping inner and outer portions such that the mesh is capable of fitting within a neonatal airway;
retaining the mesh member in the collapsed position;
delivering the stent into the airway of the neonate;
positioning the stent where desired in the airway;
implanting the stent in an expanded position in contact with the airway so that the locking projections engage the mesh of the overlapping mesh outer portion to retain the stent in the expanded state;
inserting a balloon catheter within the cylindrically shaped mesh member;
pressurizing the balloon catheter to expand the stent to a further expanded state so that the locking projections engage another section of the overlapping mesh outer portion to retain the stent in the further expanded state;
depressurizing and removing the balloon catheter from the airway; and
temporarily re-inserting and pressurizing the balloon catheter to exert sufficient outward pressure on the cylindrical stent to distend the airway of the neonate without injury, thereby encouraging the growth thereof.

21. The method of claim 20, wherein at least one of the locking projections is disposed on said mesh portion at a distance from said edge.
